(19) United States
(12) Patent Application Publication
(10) Pub. No.: US 2024/0252166 A1
(43) Pub. Date: Aug. 1, 2024

(54) SURGICAL APPLICATOR DEVICE

(71) Applicant: POLITECNICO DI TORINO, Torino (TO) (IT)

(72) Inventors: Cecilia SURACE, Torino (TO) (IT); Mariana RODRIGUEZ REINOSO, Torino (TO) (IT); Oliver GRIMALDO RUIZ, Torino (TO) (IT); Marco CIVERA, Chieri (TO) (IT); Silvia MASTROPIRRO, Bitonto (BA) (IT); Federica BERGAMIN, Torino (TO) (IT)

(21) Appl. No.: 18/290,647

(22) PCT Filed: Jul. 20, 2022
(86) PCT No.: PCT/IB2022/056685

§ 371 Date: Jan. 19, 2024

(30) Foreign Application Priority Data
Jul. 22, 2021 (IT) 102021000019484

(51) Int. Cl. A61B 17/068; A61B 17/00; A61B 17/064; A61B 17/08; A61B 17/3205

(52) U.S. Cl. CPC A61B 17/068; A61B 17/064; A61B 17/083; A61B 17/32053; A61B 2017/00367; A61B 2017/00836; A61B 2017/00862; A61B 2017/0647

(57) ABSTRACT

A T-RESAP surgical applicator device which is usable during surgeries aimed at repairing soft tissues by applying an implantable T-REM3DIE element, is configured to perforate the soft tissues to be repaired and to correctly position and insert the implantable T-REM3DIE device in the tissue to be repaired.

### CROSS-REFERENCE TO RELATED APPLICATIONS

This application is the US national stage of PCT/IB2022/056685, filed Jul. 20, 2022 and designating the United States, which claims the priority of IT 102021000019484, filed Jul. 22, 2021. The entire contents of each foregoing application are incorporated herein by reference.

### BACKGROUND OF THE INVENTION

### Field of the Invention

The present invention relates to the field of surgery, and in particular is related to a surgical applicator which allows facilitating the correct apposition of a specific type of implantable objects, used to repair soft tissues.

### Description of the Related Art

To date, applicators which are usable in the surgical field such as suture devices for laparoscopic procedures are known, corresponding to clip applicators used in endoscopic procedures to obtain the occlusion of vessels and other small tissue structures, as well as for inserting radiographic markers.

However, applicator types such as those mentioned above cannot be used to apply a specific implantable device for repairing soft tissues, called T-REM3DIE, for which—precisely—the surgical applicator of the present invention has been invented and conceived.

In the following of the present description and in the appended claims, such a term T-REM3DIE will substantially indicate a bio-compatible and bio-absorbable implantable device for repairing a soft tissue, such as a tendon and/or a ligament, comprising:
at least two bio-compatible and bio-absorbable plates comprising a first plate and a second plate made in separate pieces which can be interlocked together;
a plurality of bio-compatible and bio-absorbable connecting elements adapted to connect said first plate and said second plate together;
in which:
said first plate comprises a first surface adapted to be rested on a first side of the soft tissue to be repaired;
said second plate comprises a second surface, adapted to be rested on a second side of the soft tissue to be repaired;
each connecting element of said plurality of connecting elements comprises a first portion integral with the first plate and a second portion integral with the second plate;
said plurality of connecting elements extend from at least one of said first surface of the first plate and said second surface of the second plate to reach the other of said first surface of the first plate and said second surface of the second plate, so as to lock said first plate and said second plate in a mutually definable position, thereby acting as positioning elements of the plates with respect to the soft tissue to be repaired. To apply said T-REM3DIE on the soft tissue to be repaired, a non-

implantable perforating element is included, made in a separate piece with respect to said bio-compatible and bio-absorbable implantable T-REM3DIE device, in which said perforating device comprises a plurality of perforating elements adapted to perforate said soft tissue to form positioning paths for said plurality of connecting elements of the T-REM3DIE device itself.

It should be noted that such an implantable T-REM3DIE device—which is not the object of the present invention—is particularly useful as it allows locking the stumps of the tendons and/or ligaments in a correct mutual position, allowing them to properly set and facilitating the recovery and convalescence of the patient.

Furthermore, said T-REM3DIE device is available in a variety of formats, depending on the target tissue to be repaired.

### SUMMARY OF THE INVENTION

The task of the present invention is thus to provide a device which facilitates the precise and correct application of said T-REM3DIE, allowing both the correct positioning of said implantable device, and the reduction of the application times thereof, also reducing the intra-operative variability of the surgery.

The proposed solution consists of a mechanical device which is comparable, in some manners, to a staple gun, or stapler, configured to be able to apply the aforesaid implantable T-REM3DIE device.

Furthermore, the present invention considerably reduces the application times of the components of the T-REM3DIE biomedical system and minimizes the risk of manipulation thereof by the surgeon.

A better understanding of the invention will be obtained from the following description and with reference to the accompanying figures, showing, by way of a non-limiting example, a preferred embodiment and a variant thereof.

### BRIEF DESCRIPTION OF THE DRAWINGS

In the drawings:

FIG. 1 is a perspective view of some components of the invention, respectively of the lever (2), the connecting rod (3) and the shaft (4).

FIG. 2 is a perspective view of the piston component (6) of the invention.

FIG. 3 is a partially transparent view to show the internal thread of the piston component (6) of FIG. 2.

FIG. 4 is a perspective view of the piston component (6) and the spring (8) of the invention.

FIG. 5 is a perspective view of the component related to the lower housing (7) of the invention.

FIG. 6 is a perspective view of a detail of FIG. 5, in which the system for approaching the flaps of the soft tissue to be repaired is also shown, referred to as “stump locking means.”

FIGS. 7, 8 and 9 are side views of the assembled invention and show a gradual lowering sequence of the lever (2).

FIG. 10 is an overall perspective view of the invention, in which the return spring (8) is also visible.

FIG. 11 is a perspective view of the invention in which the stump locking means (9) are visible.

FIG. 12 is a side view of the invention, related to a section according to the vertical plane of symmetry.

FIG. 13 is a perspective view of a variant of the lower support (7′).

FIG. 14 is a view of a blade (74) usable with said variant of the lower support (7′).

FIG. 15 is a perspective view of the variant of the lower support (7′), in which the system for approaching the flaps of soft tissue flaps to be repaired is also shown, referred to as “stump locking means.” FIG. 16 is a perspective view of the variant of the lower support (7′) with a blade (74) inserted in the special slot (73).

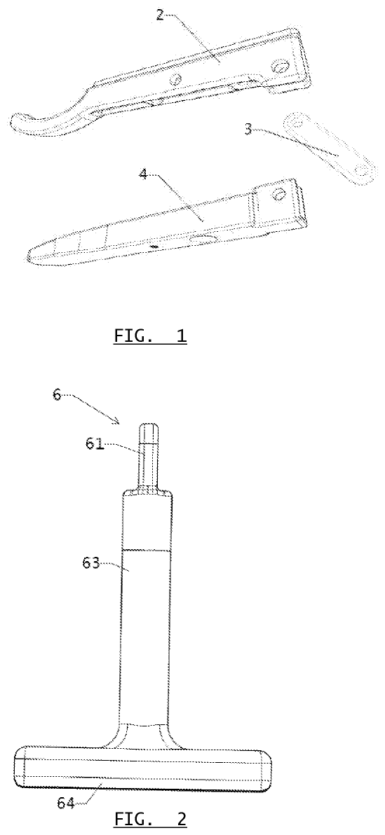

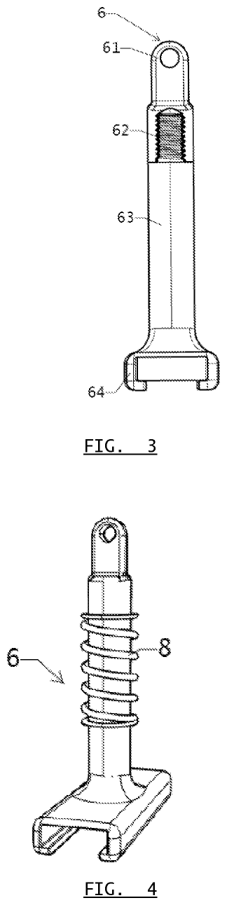

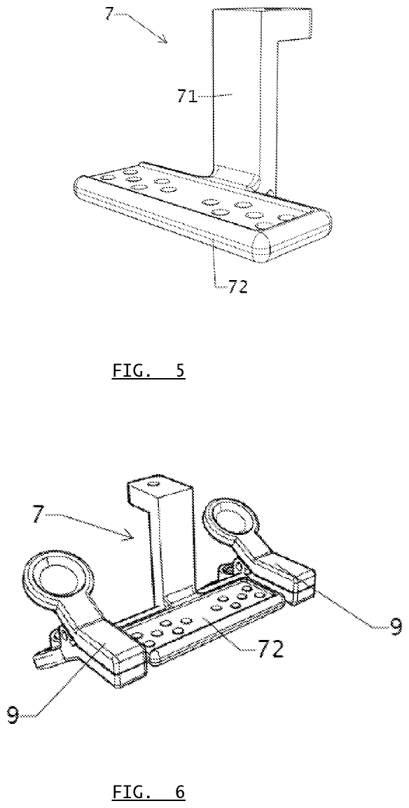

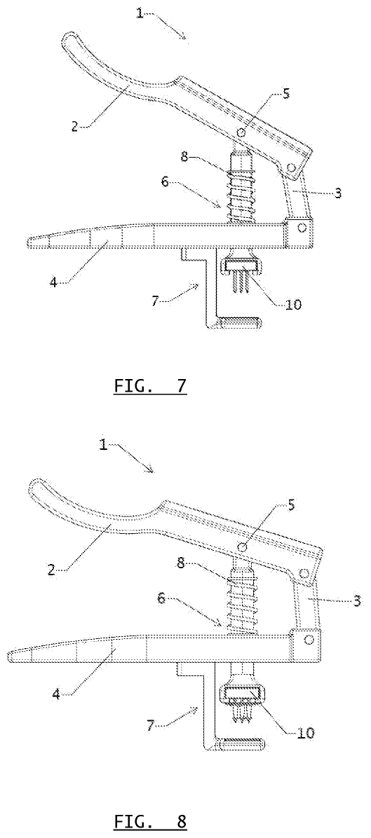

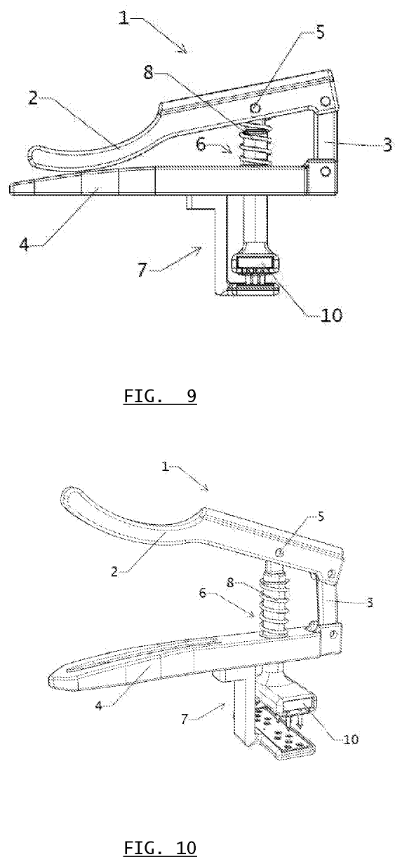

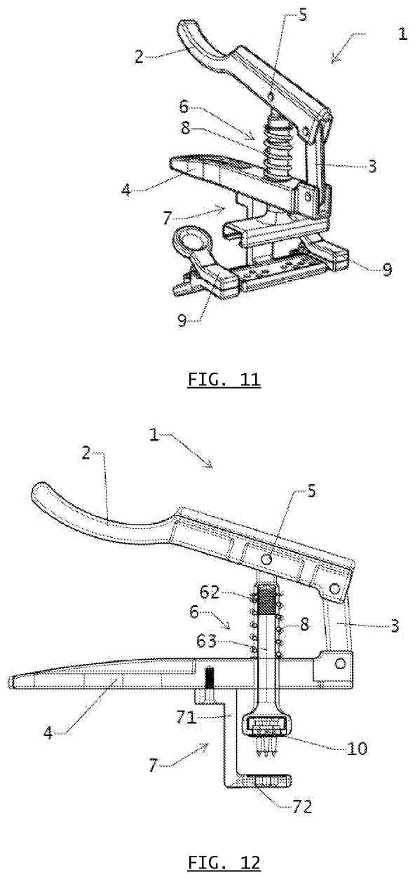

### DETAILED DESCRIPTION

With reference to the aforementioned figures, the surgical applicator of the present invention, which will hereinafter also be referred to as T-RESAP (Tendon Repair Surgical Applicator), is a tool conceived and designed to be directly couplable with the implantable T-REM3DIE device described above.

Said applicator, which is usable during surgeries aimed at repairing soft tissues, is not in itself an implantable tool, but is useful for guiding the perforation, the correct positioning and the insertion of the implantable T-REM3DIE device in the tissue to be repaired.

In other words, the invention allows guiding and facilitating the process of inserting and installing the T-REM3DIE device.

In a preferred but non-limiting embodiment, T-RESAP applicator device according to the invention (1) comprises the following components:
an upper handle, or lever (2);
a lower component, or shaft (4);
means (3) for connecting the lever (2) to the shaft (4) so as to form a second kind lever;
a piston (6), adapted to transform the rotation of the handle (2) with respect to the shaft (4) into a translational movement, and configured to house a perforating plate (10) of the T-REM3DIE device, together with a first plate of the latter;
a lower support (7) configured to house a second plate of the T-REM3DIE device;
means (5) for coupling the lever (2) to the piston (6);
elastic means (8) for returning the piston (6), and thus the lever (2), to the initial position with respect to the shaft (4).

Optionally, means (9) for temporarily fixing the stumps of the soft tissue to be repaired can be included at the lower support (7).

The T-RESAP applicator object of the present invention is thus formed by the set of components listed above; said components interact with each other, allowing the insertion and guided positioning of the T-REM3DIE device in the target tissue.

In summary, the T-RESAP applicator has the configuration of a second kind lever, where the application point of the action force F_{A}, i.e., the force exerted by the operator on the lever (2), is farther from the fulcrum than the point where the resistant force F_{R} is found, i.e., the force to be exerted for penetrating into the tendon tissue and also inclusive of any elastic resistance due to the return spring (8). It is therefore always an advantageous lever because the action force arm b_{A} is greater than the resistant force arm b_{R}.

The force on the lever (2) translates into the gradual lowering of the piston (6) so as to move it by a precise amount in the vertical direction.

In practice, the surgeon tightens the movable lever (2) and the fixed shaft (4) of the applicator together with one hand, activating the piston (6) which transmits the movement thereof to the perforating plate (10).

The downward excursion of the piston (6) allows the gradual approach of the T-REM3DIE housed therein; the force imposed by the applicator, when inserting the T-REM3DIE into the soft tissue, is facilitated by the ratio of the arms of the second kind lever and is accurately controlled by the operator's hand.

Therefore, the technical solution which is described according to the invention is based on the generation of a force which can be modulated by the experience of the operator, sufficient to perforate the tendon (or other soft tissue) through a mechanism which uses a lever and a piston.

Thereby, exerting a certain force on the lever (2) of the T-RESAP applicator, the movement of the piston (6) is imposed, which gradually approaches the target tissue for an accurate and correct positioning and installation of the T-REM3DIE device.

During the use of the applicator according to the present invention, the T-REM3DIE to be implanted is partly housed in the piston (6) of the applicator and partly in the housing of the lower support (7) of the same applicator; in the preferred embodiment described so far, the piston (6) and the lower support (7) are both fixable with a screw-nut type coupling to be interchangeable (62, 71).

According to the invention, the housing (64) of the first plate and of the perforating plate of the T-REMEDIE device is placed in the lower area of the piston (6) and has the task of holding these two elements of the T-REM3DIE, keeping them in a precise position during the application; the non-implantable perforating plate (10), provided with a plurality of sharp perforating elements, is inserted inside a groove present in the housing (64) present in the piston (6), while the first implantable plate, with the pins, is rested below the perforating plate (10) inserting the pins thereof into the plurality of sharp perforating elements; thereby said implantable plate is correctly placed and remains in position by friction.

The second plate of the T-REM3DIE, or complementary plate, provided with holes for housing the respective pins of the first plate, is rested on the special housing seat (72) forming part of the lower support (7).

Once the two components of the T-REM3DIE, which are not the subject of the present invention, are interlocked, they remain in situ in the soft tissue to join the two flaps thereof, while the perforating plate (10) is extracted when the T-RESAP applicator is opened for extraction.

In a preferred, but non-limiting, embodiment, the lever (2) has a curvature to allow a more ergonomic grip and ensure an easier positioning of the hand.

So as to obtain a straight translation of the piston (6) to ensure the correct perforation of the soft tissue and the correct coupling of the two parts of the T-REM3DIE, in the preferred embodiment described, it is envisaged to connect the lever (2) to the shaft (4) by means of a connecting rod (3) the ends of which are pivoted to the corresponding ends of the lever (2) and the shaft (4), in which the lever (2) and the piston (6) are hinged together by means of a simple pin (5). To this end, said shaft (4) is provided with a transverse guide hole for the piston (6), in which the latter slides with straight motion.

A similar result could be obtained by using other connecting means between lever (2) and shaft (4) and between lever (2) and piston (6). For example, the lever (2) could directly pivot to the shaft (4) by means of a simple end pin, envisaging in such a case that the pin (5) constraining the piston (6) to the lever (2) is housed in an elongated hole of the lever itself instead of in a round hole, however obtaining a straight translation of the same piston (6) with respect to the shaft (4).

In the preferred embodiment described so far, a return spring (8) is included which has the function of returning the applicator device to the initial configuration, after having completed the insertion of the T-REM3DIE device on the target tissue.

The spring (8) is preferably a thrust spring placed around the plunger (63) of the piston (6), between the lever (2) and the shaft (4).

The approach of the lever (2) to the shaft (4) causes the piston (6) to lower and the spring (8) to compress with respect to the equilibrium condition. The energy thus accumulated by the spring (8), once the needles present on the perforating plate (10) have penetrated the target tissue and the consequent insertion of the T-REM3DIE, is such as to exert a force directed upwards so that, once the operator stops tightening the lever (2) on the shaft (4), the piston (6) lifts and the return to the starting position occurs with the opening of the T-RESAP applicator.

Advantageously, this allows an easier removal of the perforating plate (10) with the needles and an automatic opening of the applicator itself.

The elastic constant of the return spring (8) was chosen based on the mass of the lever (2), the piston (6), the perforating plate (10) and the stroke of the piston (6).

At the housing present in the lower support (7) for the second plate of the T-REM3DIE, means (9) for temporarily fixing the stumps of the soft tissue to be joined are included. Such means, which are an approach system for the two parts of the soft tissue to be repaired, preferably consist of two specular rods, ending with clips which directly staple the target tissue without causing micro-injuries.

Said clips are placed at the ends of the housing seat (72) present in the lower support (7) so as to retain the target tissue from the ends, leaving space for the application of the T-REM3DIE.

The invention is preferably made entirely of 316L stainless steel; thereby it is possible to break down the instrument into the parts thereof and individually sterilize the individual components.

Furthermore, the geometry of the device is small, resulting in reduced invasiveness in the operating field.

In a variant (not shown), the interchangeable parts of the piston (6) and the lower support (7) for the housing of the T-REM3DIE device can also be made of high-strength polymer material, the choice of material being related to the prospects of use:
High-strength polymer material to allow a single use (disposable mode);
Metal material (316L stainless steel) to be sterilizable and used for a given number of operations before being replaced.

As mentioned, the present invention allows for an easier and faster application of the T-REM3DIE device, guiding the installation procedure in the target tissue.

Furthermore, the housing means of the plates, both the upper ones (63, 64) of the piston (6) and the lower ones (72) of the lower support (7) are interchangeable in order to be able to use housing elements of dimensions always suitable for the various T-REM3DIE plates to be applied in different cases.

A further advantage is that the devised device includes few application steps, allowing the reduction of inter-operator variability and reproducibility for any body district where the T-REM3DIE device can be applied.

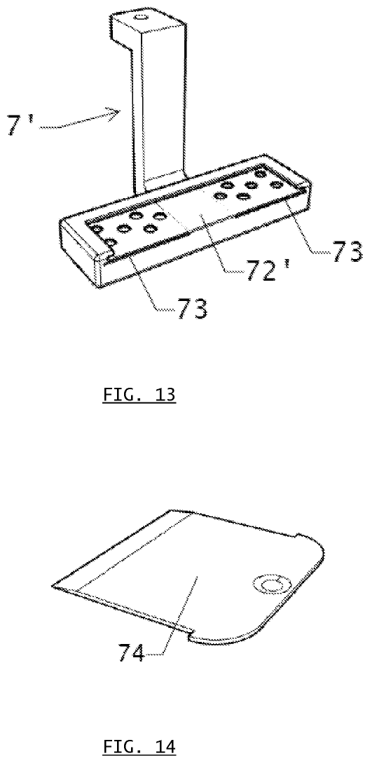

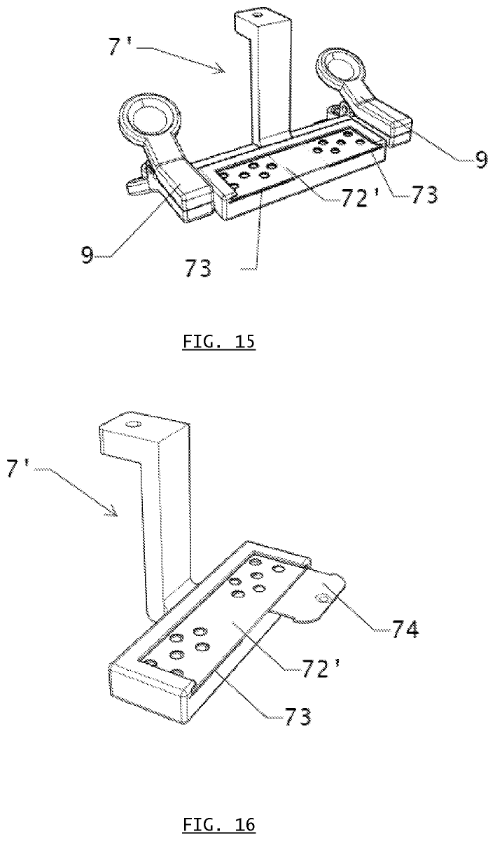

In a variant of the invention, shown in FIGS. 13 to 16, the lower support (7′) is provided with a different housing seat (72′) of the complementary plate of the T-REM3DIE device, which in this case can be inserted frontally.

According to such a variant, there is at least one slot (73) below said housing seat (72′) in which a blade (74) can be inserted in a frontal position.

Said blade (74) has the function of cutting the perforating tips of the T-REM3DIE device, once applied to the soft body to be repaired.

According to the variant of the invention, which is described by way of non-limiting explanation, there are two of said slots (73), in which respective blades (74) are inserted.

1. A T-RESAP surgical applicator device which is usable during surgeries aimed at repairing soft tissues by applying an implantable T-REM3DIE element, wherein said device is configured to perforate the soft tissues to be repaired and to correctly position and insert the implantable T-REM3DIE device in the tissue to be repaired.

2. A device according to claim 1, wherein the device comprises:
an upper handle, or lever;
a lower component, or shaft;
end means for connecting the lever to the shaft so as to form a second kind lever;
an element, or piston, articulated with respect to the lever and sliding transversely with respect to the shaft, adapted to transform the approaching movement of the lever to the shaft into a straight translational movement; said piston being configured to support and actuate a soft tissue perforating plate and a first plate with pins of the T-REM3DIE;
a lower support configured to house a second plate of the T-REM3DIE;
articulation means between the lever and the piston elastic means for returning to the initial position of the lever, placed between the lever itself and the shaft.

3. The device according to claim 2, further comprising means for temporarily fixing the stumps of the soft tissues to be repaired.

4. The device according to claim 2, wherein the device has the configuration of a second kind lever, where the application point of the action force F_{A}, exerted by the operator on the lever, is farther from the fulcrum than the point where the resistant force F_{R} to be exerted on the piston for the perforation of the soft tissue is found.

5. The device according to claim 2, wherein the device is configured so that the force on the lever, exerted by the operator, results into the gradual lowering of the piston, thus allowing the piston to be moved by a precise amount in the vertical direction, said movement of the piston being modulable according to the force exerted on the lever.

6. The device according to claim 2, wherein the connecting means between lever and shaft and the articulation means between lever and piston are such as to allow a perfectly straight movement of the piston inside the guide means thereof, with respect to the shaft.

7. The device according to claim 2, wherein the housing means of the T-REM3DIE plates, both that being integral with the piston and that of the lower support, are interchangeable.

8. The device according to claim 2, wherein the lower support is provided with a housing seat for the complementary plate of the T-REM3DIE device with frontal insertion, and at least one slot for the insertion of a respective blade adapted to cut the perforating elements of said T-REM3DIE device once positioned on the soft body to be repaired.

9. The device according to claim 1, wherein the device is made entirely of 316L stainless steel, or of another rigid material adapted to the purpose.

10. The device according to claim 1, wherein the parts forming the housing for the two T-REM3DIE plates are made of high-strength polymer material for disposable use, while the remaining parts are made of 316L stainless steel.

11. The device according to claim 2, wherein said end means, for connecting the lever to the shaft, consist of a connecting rod the ends of which are pivoted to the corresponding ends of the lever and shaft, wherein the lever and the piston are hinged together by means of a simple pin.

12. The device according to claim 2, wherein said end means, for connecting the lever to the shaft, consist of a simple end pin by means of which the lever is directly hinged to the shaft; in this case a pin constraining the piston to the lever being included, housed in an elongated hole of the lever itself instead of in a round hole, however obtaining a straight translation of the same piston with respect to the shaft.

13. The device according to claim 11, wherein said shaft is provided with a transverse guide hole for the piston, in which the piston slides with straight motion during the movement of the lever with respect to the shaft.

14. The device according to claim 2, wherein said elastic means for returning to the initial position of the lever consist of a compression spring configured so that the approach of the lever to the shaft causes the piston to lower and the spring to compress with respect to the equilibrium condition; the energy thus accumulated by the spring being such as to exert a force directed upwards so that, once the operator stops tightening the lever on the shaft, the piston lifts and the return to the starting position occurs with the opening of the T-RESAP applicator.

15. The device according to claim 3, wherein the device has the configuration of a second kind lever, where the application point of the action force F_{A}, exerted by the operator on the lever, is farther from the fulcrum than the point where the resistant force F_{R} to be exerted on the piston for the perforation of the soft tissue is found.

16. The device according to claim 15, wherein the device is configured so that the force on the lever, exerted by the operator, results into the gradual lowering of the piston, thus allowing the piston to be moved by a precise amount in the vertical direction, said movement of the piston being modulable according to the force exerted on the lever.

17. The device according to claim 16, wherein the connecting means between lever and shaft and the articulation means between lever and piston are such as to allow a perfectly straight movement of the piston inside the guide means thereof, with respect to the shaft.

18. The device according to claim 17, wherein the housing means of the T-REM3DIE plates, both that being integral with the piston and that of the lower support, are interchangeable.
